(19) United States
(12) Patent Application Publication
(10) Pub. No.: US 2014/0371326 A1
(43) Pub. Date: Dec. 18, 2014

(54) FOOD COMPOSITION FOR PREVENTING OBESITY, PHARMACEUTICAL COMPOSITION FOR TREATING OBESITY, AND ANIMAL MEDICINE FOR TREATING OBESITY, CONTAINING GINGERNONE A

(71) Applicant: SNU R&DB Foundation, Seoul (KR)

(72) Inventors: Ki Won Lee, Seoul (KR); SuJin Seok, Daejeon (KR)

(73) Assignee: SNU R&DB FOUNDATION, Seoul (KR)

(21) Appl. No.: 14/317,880

(22) Filed: Jun. 27, 2014

Related U.S. Application Data
(63) Continuation of application No. PCT/KR2012/011551, filed on Dec. 27, 2012.

(30) Foreign Application Priority Data
Dec. 29, 2011 (KR) 10-2011-0146238

(51) Int. Cl. A23L 1/30; A61K 31/12

(52) U.S. Cl. CPC A23L 1/30; A61K 31/12
USPC 514/679; 568/325

(57) ABSTRACT

Disclosed is a composition comprising gingerenone A as an active ingredient. The composition includes a food composition for preventing obesity, a pharmaceutical composition for treating obesity and a medicine for treating animal obesity. Since the composition includes gingerenone A, which inhibits expression of the important transcriptional factors C/EBPα and PPARγ, expressed upon adipocyte differentiation, as well as FAS protein expression, the composition has superior potential for obesity prevention or treatment.

### CROSS-REFERENCE TO RELATED APPLICATIONS

This application is a continuation of International Application No. PCT/KR2012/011551 filed on Dec. 27, 2012, which claims priority to Korean Application No. KR10-2011-0146238 filed on Dec. 29, 2011, which applications are incorporated herein by reference.

### TECHNICAL FIELD

The present invention relates to a food composition for preventing obesity, a pharmaceutical composition for treating obesity and a medicine for treating animal obesity comprising gingerenone A as an active ingredient.

### BACKGROUND ART

According to a report, two thirds of people in England and the USA are overweight, while a quarter of people in these countries are obese. These statistics highlight the growing problem of obesity as a serious medical problem across the globe. According to an annual report from the Ministry of Health & Welfare in Korea, adult obesity rates in 2007 had increased by more than 10% when compared to 1998. Childhood obesity also increased greatly.

Obesity is caused by overeating and a lack of physical activity, as well as genetic and other factors. Among these factors, high-calorie foods common to a typical Western diet are a primary contributing factor. Obesity affects both health and quality of life, and contributes to the development of diseases including type 2 diabetes, high blood pressure, hypercholesterolemia, and cardiovascular disorders. The prevalence of these metabolic diseases related to obesity is gradually increasing, resulting in greater societal costs.

Diabetes is characterized by chronic hyperglycemia induced by dysfunctional insulin signaling, and causes a variety of metabolic problems. The disease is classified into insulin-dependent type I diabetes mellitus and insulin-independent type II diabetes. Of these, the recent upsurge in type II diabetes has been linked to increasing rates of obesity.

In adults, high blood pressure is defined as a systolic blood pressure of 140 mmHg or more, in addition to a diastolic blood pressure of 90 mmHg or more. In Korea, high blood pressure is generally more prevalent than in other countries, with more than 30% of Korean adults suffering from the condition. Chronic high blood pressure can lead to a number of medical complications including coronary artery disease, stroke, and renal failure. Many of these complications cause serious ongoing health problems and can threaten the life of patients.

Hypercholesterolemia refers to a state in which cholesterol levels exceeding normal values are present in the blood due to excess cholesterol consumption or metabolic disorders. Hypercholesterolemia can lead to complications including angina, heart attack, and stroke.

Due to the strong link between obesity and metabolic diseases, novel and effective methods for the prevention and treatment of obesity are urgently needed. To date, drugs for the treatment of obesity have included satiety activators, fat absorption inhibitors and psychotropic appetite suppressants, which are each classified by their functional target pathway.

Satiety activators suppress the reabsorption of serotonin and noradrenalin which control appetite in the brain and, as such, decrease appetite and increase energy consumption by increasing the basal metabolic rate. Reductil, a representative example of a satiety activator, contains the active ingredient sibutramine. Due to its unacceptable side effects on the cardiovascular system, reductil is no longer available.

Fat absorption inhibitors suppress the action of lipase, an enzyme that contributes to fat absorption, resulting in a net increase in fat excretion from the body. Orlistat is a representative fat absorption inhibitor.

Psychotropic appetite suppressants lower the sensation of appetite by promoting generation of the neurohormones norepinephrine and dopamine, and thereby control appetite signaling in the brain. Psychotropic appetite suppressants contain active ingredients such as phendimetrazine and phentermine.

All currently-available obesity treatment drugs cause a variety of side effects. The Ministry of Food and Drug Safety halted sales of sibutramin and recommended its voluntary recall in October 2010. Sibutramin is subsequently no longer on the market. Xenical has been purported to have fewer side effects, although documented evidence includes the appearance of steatorrhea, flatulence, abdominal swelling and other symptoms. Recently, the number of patients reporting kidney failure while taking xenical has been increasing.

The considerable side effects associated with current obesity drugs represent a clear unmet medical need. Accordingly, interest in obesity treatment drugs derived from natural ingredients has been rising. A report by the Korean Intellectual Property Office states that the number of patent applications related to obesity treatment drugs derived from natural substances increased from five in 2000 to forty-two in 2008, indicating a significant increase in the amount of research in this area.

Over the decade spanning 1998 to 2008, patents for obesity treatment drugs derived from natural substances based on traditional oriental medicine have been used in consumer products. Natural substances including plants such as oriental herbal constituents, green tea, ginseng, pine needles, brown algae, as well as minerals, microorganisms, and other substances have all been used. A representative example is an obesity treatment drug containing genistein. Genistein is a natural compound abundant in soybeans (M. Zhang et al., Phytother Res. 23(5):713-8, 2009; H. J. Park et al., J. Nutr. Biochem. 20(2):140-8, 2009).

However, the understanding of the currently-known natural substances and the detection of new natural substances with a higher efficacy than genistein for the prevention and treatment of obesity is still needed.

Korean Patent No. 10-0588469 entitled “Composition for Prevention and Treatment of Obesity Comprising Shogaol from Zingiber Officinale”, reports that shogaol, a compound extracted from ginger, inhibits the generation of adipocytes and promotes fat excretion. In addition, a functional food and a pharmaceutical composition comprising shogaol are disclosed in the patent literature. However, to the best of our knowledge, the patent literature does not contain any references to the potential obesity preventive and treatment effects of gingerenone A.

Furthermore, in Korean Patent Application No. 10-2011-0036333 entitled “Herb Medicine Extract Having Anti-obesity Activity and Manufacturing Method Thereof”, a method to efficiently extract the active fraction from the zingiberis rhizome, which inhibits adipocyte differentiation and inhibits fat accumulation, as well as its obesity preventive effects, have been disclosed. However, the literature does not appear to contain references to the obesity preventive and treatment effects of gingerenone A.

### SUMMARY

The present invention has been made in view of the above listed problems, and it is an objective of the present invention to provide a food composition for preventing obesity, a pharmaceutical composition for treating obesity and a medicine for treating animal obesity, using a new active natural material exhibiting anti-obesity effects.

In accordance with an aspect of the present invention, the above and other objects can be accomplished by the provision of a food composition for preventing obesity comprising gingerenone A, represented by Formula 1, as an active ingredient.

In accordance with another aspect of the present invention, provided is a pharmaceutical composition for treating obesity or a medicine for treating animal obesity, comprising gingerenone A, represented by Formula 1, as an active ingredient:

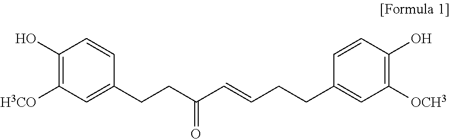 [SMILES: COc1cc(CC/C=C/C(=O)CCc2ccc(O)c(OC)c2)ccc1O]

### BRIEF DESCRIPTION OF DRAWINGS

The above and other objects, features and other advantages of the present invention will be more clearly understood from the following detailed description in conjunction with the accompanying drawings, in which:

FIG. 1 illustrates a process of synthesizing gingerenone A utilized in the present invention. In detail, gingerenone A is synthesized by hydrogenating and dehydrating curcumin, which is extracted from turmeric root and is a natural dye.

FIG. 2 illustrates viability of 3T3-L1 cells after treatment with gingerenone A. When 3T3-L1 cells are treated with 40 μM gingerenone A, cytotoxicity is not observed at 24 and 48 hours after treatment.

FIG. 3 illustrates the inhibitory effects on fat accumulation exhibited by gingerenone A. Lane 1 is a negative control in which the cells are not differentiated. Lane 2 is a control in which cells have differentiated due to MDI treatment. Lane 3 was treated with MDI and 10 μM gingerenone A. Lane 4 was treated with MDI and 20 μM gingerenone A. Lane 5 was treated with MDI and 40 μM gingerenone A.

FIG. 4 illustrates the C/EBPα, PPARγ and FAS protein inhibitory effects of gingerenone A. Lane 1 is a negative control in which cells are not differentiated. Lane 2 is a control in which cells are differentiated due to MDI treatment. Lane 3 was treated with MDI and 10 μM of gingerenone A. Lane 4 was treated with MDI and 20 μM of gingerenone A. Lane 5 was treated with MDI and 40 μM gingerenone A.

FIG. 5 illustrates the comparative C/EBPα, PPARγ and FAS protein inhibitory effects of gingerenone A and gingerol. Lane 1 is a negative control in which cells are differentiated. Lane 2 is a control in which cells are treated with MDI. Lane 3 was treated with MDI and 10 μM of gingerenone A. Lane 4 was treated with MDI and 20 μM of gingerenone A. Lane 5 was treated with MDI and 40 μM gingerenone A. Lane 6 was treated with 40 μM of gingerol.

### DETAILED DESCRIPTION

Hereinafter, the technical solution of the present invention is described in detail.

The present inventors investigated gingerenone A, a biologically active material, present in ginger (zingiber officinale roscoe), to identify a biologically active material exhibiting obesity suppressive effects.

In general, ginger extract is comprised of the biologically active compounds gingerol and [6]-shogaol. It was reported that both gingerol and [6]-shogaol have anti-inflammatory effects (Grzanna et al., J. Med. Food 8(2):125-132, 2005; Lantz et al., Phytomedicine 14(2-3):123-8, 2007), anti-cancer effect (Katiyar et al., Cancer research 56(5):1023-1030, 1996; Surh, Food Chem. Toxicol. 40(8):1091-7, 2002), antioxidant effect (Eguchi et al., Free Radic. Res. 39(12):1367-1375, 2005; Masuda et al., BioFactors 21(1-4):293-6, 2004).

Gingerenone A is a relatively newly-discovered material from ginger extract, and to date, there have been few reports on its biological activity. Gingerenone A has a diarylheptenone structure, and its shorthand chemical formula is C₂₁H₂₄O₅.

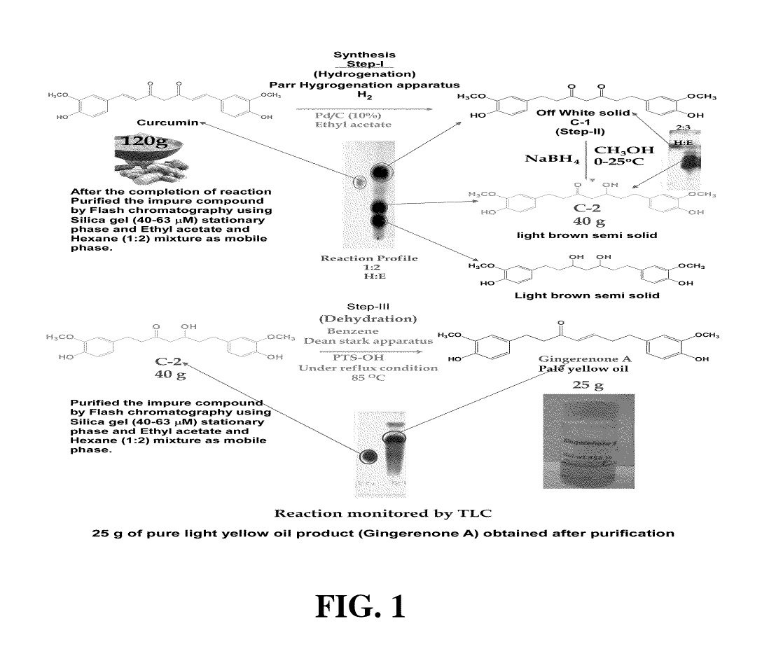

Gingerenone A as specified in the present invention may be synthesized by hydrogenating and dehydrating curcumin, which is a natural dye found in the turmeric root, according to the process illustrated in FIG. 1.

When stem cells differentiate into adipocytes in the human body, the differentiation phases are classified into three steps, namely, the commitment, mitotic clonal expansion, and terminal differentiation steps. In the commitment step, stem cells differentiate into pre-adipocytes. In the mitotic clonal expansion step, proliferation causes the number of cells to increase. Finally, in the terminal differentiation step, cellular fat content increases.

In the mitotic clonal expansion step, each pre-adipocyte proliferates two to four times, and this step is regarded as essential for fat cell differentiation (Q. Q. Tang et al., Proc Natl Acad Sci USA, 100(1):44-9, 2003). In the mitotic clonal expansion step, C/EBPβ (CCAAT/enhancer-binding protein β) is an important transcriptional factor. It was reported that if C/EBPβ function is inhibited, differentiated adipocytes will not form in mice (Q. Tang et al. Proc Natl Acad Sci USA, 100(3):850-5, 2003).

In the terminal differentiation step, the fat content of differentiated adipocytes increases. This step is promoted by important transcriptional factors, including C/EBPα (CCAAT/enhancer-binding protein α) and PPARγ (peroxisome proliferator-activated receptor γ) (E. D. Rosen et al., Mol Cell, 4(4):611-7, 1999). The differentiation of pre-adipocytes into adipocytes is therefore governed by cross-regulation between the two cell types (Z. Wu et al., Mol Cell, 3(2):151-8, 1999).

FAS (fatty acid synthase) is an enzyme that promotes the synthesis of cellular neutral fat. FAS expression is controlled by the PPARγ transcriptional factor. When FAS expression is downregulated, food intake and bodyweight decrease (Loftus et al., Science, 288((5475):2379-2381, 2000).

For the present invention, it has been confirmed that gingerenone A controls fat accumulation, and the expression of important transcriptional factors (C/EBPα and PPARγ) which are expressed upon adipocyte differentiation, and FAS protein expression. It was confirmed that gingerenone A suppresses cellular fat accumulation, as well as the expression of C/EBPα, PPARγ and FAS proteins.

In addition, the relative suppression of C/EBPα, PPARγ and FAS expression levels by gingerol and gingerenone A were compared. Gingerenone A exhibited superior obesity preventive and treatment effects when compared to gingerol.

Gingerenone A, as an active ingredient described in the present invention, may be produced via synthetic methods or extracted from ginger.

The present invention describes a food composition for preventing obesity, comprised of gingerenone A as an active ingredient. The food composition comprises, preferably, 0.000001 to 50% by weight of gingerenone A, based on a 100% total weight of the food composition. When a food composition comprises less than 0.000001% by weight of gingerenone A, based on a total 100% weight of the food composition, obesity prevention and treatment effects are too low. In contrast, when a food composition comprises more than 50% by weight of gingerenone A, based on a total 100% weight of the food composition, cost-efficiency is low.

A food composition including the present invention for preventing obesity may comprise at least one of the following, preferably, meat, grains, caffeine beverages, beverages, chocolate, bread, snacks, biscuits, pizza, jelly, noodles, gums, ice cream, alcoholic beverages, vitamin supplements and other healthy supplement foods, although the present invention is not limited thereto.

The present invention provides a pharmaceutical composition for treating obesity or a medicine for treating animal obesity, comprising gingerenone A as an active ingredient.

The amount of gingerenone A present in a pharmaceutical composition for treating obesity or a medicine for treating animal obesity may be dependent on the usage of other prevention and treatment agents, the conditions of patients and animals, other disease types present, and their severity.

A pharmaceutical composition for treating obesity or a medicine for treating animal obesity of the present invention comprises, preferably, 10 μM to 1 mM by weight of gingerenone A, based on a 100% total weight of the pharmaceutical composition or medicine, although the present invention is not limited thereto.

A pharmaceutical composition for treating obesity or a medicine for treating animal obesity of the present invention may comprise an active ingredient. The pharmaceutical composition for treating obesity or a medicine for treating animal obesity of the present invention may further comprise pharmaceutically acceptable carriers, dilutes or excipients. The pharmaceutically acceptable carriers, excipients or dilutes may comprise at least one selected from lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil. In addition, when the pharmaceutical composition for treating obesity or the animal medicine of the present invention is a drug, the drug may further comprise fillers, anticoagulants, lubricants, humectants, spices, emulsifiers, preservatives or similar substances.

The formulation of a pharmaceutical composition for treating obesity or a medicine for treating animal obesity of the present invention may be of a preferable type in accordance with the way it is used. In particular, the formulation may be prepared according to a method known in the art in order to provide fast, continuous or delayed release of the medicine after administering the formulation to mammals. Examples of the formulation may comprise at least one selected from plasters, granules, lotions, liniments, lemonades, aromatic water, powders, syrups, ophthalmic ointments, liquids and solutions, aerosols, extracts, elixirs, ointments, fluid extracts, emulsions, suspensions, decoctions, infusions, ophthalmic solutions, tablets, suppositories, injections, spirits, cataplasms, capsules, creams, troches, tinctures, pastes, pills, soft gelatin capsules and hard gelatin capsules.

A dosage of a pharmaceutical composition for treating obesity or a medicine for treating animal obesity of the present invention may be varied, according to the manner in which it is administrated, as well as animal age, gender, weight, disease severity, and other factors. For example, a medicine for preventing or treating obesity involving the present invention may be administered more than once a day in an amount between 0.1 and 100 mg/kg, based on body weight. However, these dosages are exemplary only and it is not intended to be limiting of the dosage. The dosage may be changed by the prescription of a physician, according to animal conditions.

As apparent from the evidence described in this document, the present invention advantageously provides a food composition for preventing obesity, or a pharmaceutical composition for treating obesity and a medicine for treating animal obesity, comprising gingerenone A, as an active ingredient, exhibiting suppressive effects on fat accumulation, suppressive effects on C/EBPα and PPARγ proteins expressed upon adipocyte differentiation and suppressive effects on FAS protein expression controlling neutral fat synthesis.

### EXAMPLES

Hereinafter, the present invention will be described in more detail with reference to the following Examples. The scope of the present invention is not limited to the following Examples and covers modifications of the technical spirit substantially equivalent thereto.

### Experimental Example 1

### Synthesis of Gingerenone A from Curcumin

Gingerenone A utilized in the present invention was synthesized by hydrogenating and dehydrating curcumin, which is a natural material extracted from turmeric root according to the process shown in FIG. 1. A constitutional formula of the synthesized compound is identical to the above Formula 1.

### Experimental Example 2

### Measurement of Effects of Gingerenone A on Viability of 3T3-L1 Cells

In Experimental Example 2, in order to measure the toxicity of 313-L1 cells in the presence of varying concentrations of gingerenone A, MTT3-(4,5-Dimethylthiazol-2-yl)-2,5diphenyltetrazolium bromide) analysis was conducted.

First, after plating 313-L1 cells into 96 well plates, the cells were cultured until the 313-L1 cells became confluent. Thereafter, the 313-L1 cells were treated with gingerenone A at concentrations of 10, 20 and 40 μM. After culturing for 24 and 48 hours, MTT was added to the cultured cells to a concentration of 0.5 mg/ml. The treated cells were cultured for 4 hours at 37° C. in a 5% CO₂ incubator. Thereafter, the media was removed and 200 μl of DMSO (dimethylsulfoxide) was added to measure absorbance at a wavelength of 570 nm using an absorbance reader (Versa Max, Molecular Device, California, USA). Cell viability was determined as a percentage with respect to a control treated with only MDI (0.5 mM of isobutyl-methylxanthine, 1 μM of dexamethasone and 10 μg/ml of insulin).

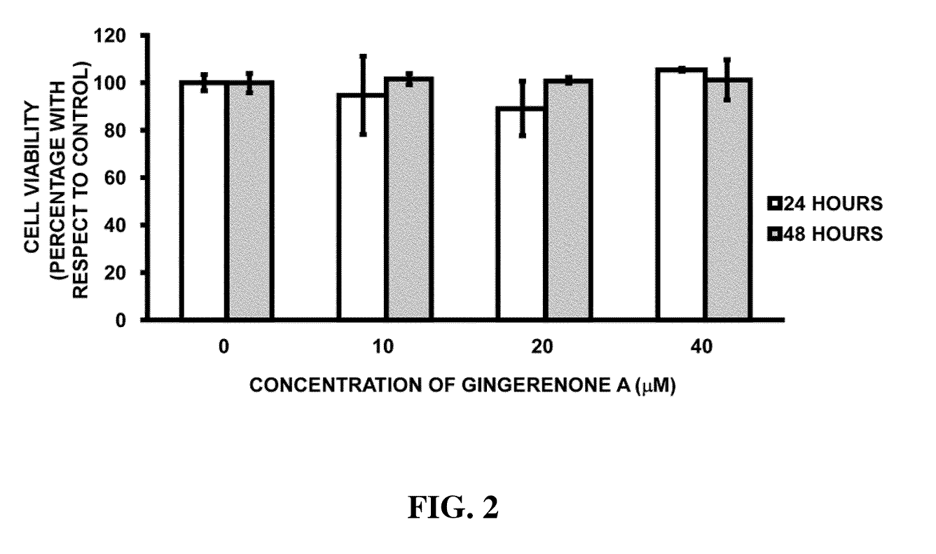

FIG. 2 shows the MTT analysis result. When 3T3-L1 pre-adipocytes were treated with 10, 20 and 40 μM gingerenone A, cytotoxicity was not observed.

### Experimental Example 3

### Measurement of Suppressive Effects of Gingerenone A on Fat Accumulation

In Experimental Example 3, to analyze the suppressive effects of gingerenone A on fat accumulation, oil red 0 staining was performed after inducing differentiation of the pre-adipocytes (K. Tobe et al., FEBS Lett, 215(2):345-9, 1987).

The 3T3-L1 cells (ATCC, Gaithersburg, Md.), representing pre-adipocytes, were plated into 24-well plates. The plated 3T3-L1 cell line was cultured in DMEM (Dulbecco-modified Eagle medium, WELGENE) with 10% FBS (fetal bovine serum) and 10 ml/L, of antibiotic-antimycotic agent at 37° C. in a 5% CO2 incubator (MCO-15AC, SANYO E&E, Europe) until the cells reached 100% confluence.

When the 313-L1 cells reached 100% confluence, the cells were further cultured for 2 days in DMEM medium, in which MDI was added, with 10 ml/L of antibiotic-antimycotic agent and 10% fetal bovine serum (FBS). The media was replaced with DMEM containing 10 ml/L of insulin, 10% FBS and 10 ml/L of antibiotic-antimycotic agent for another 2 days. Thereafter, the media was changed to DMEM medium only comprising 10 ml/L of antibiotic-antimycotic agent and 10% FBS. After further culturing the cells for 2 days, completely differentiated adipocytes, namely, mature adipocytes, were formed.

When 313-L1 cells were differentiated in the presence of MDI, gingerenone A was added at concentrations of 10, 20 and 40 μM to the cell culture media every third day from the day of MDI addition. Gingerenone A was dissolved in DMSO. After culturing for 6 days, the media was removed when the differentiation of the cells had finished. Fat globules present in the differentiated cells were stained. For lipid droplets staining, the cells were stained by 400 μl of 3% to 4% formaldehyde. After fixing for 1 hour, the formaldehyde was removed. Thereafter, the fixed cells were washed with 60% isopropanol and the washing solution was removed by suction. Thereafter, the plates containing the washed cells were stained with 400 μl of oil red O solution for at least 1 hour. After removing the oil red O solution, the stained cells were washed four times with PBS and then the oil red 0 stain was eluted with 500 μl of 100% isopropanol. The concentration was measured at a wavelength of 515 nm using an absorbance reader.

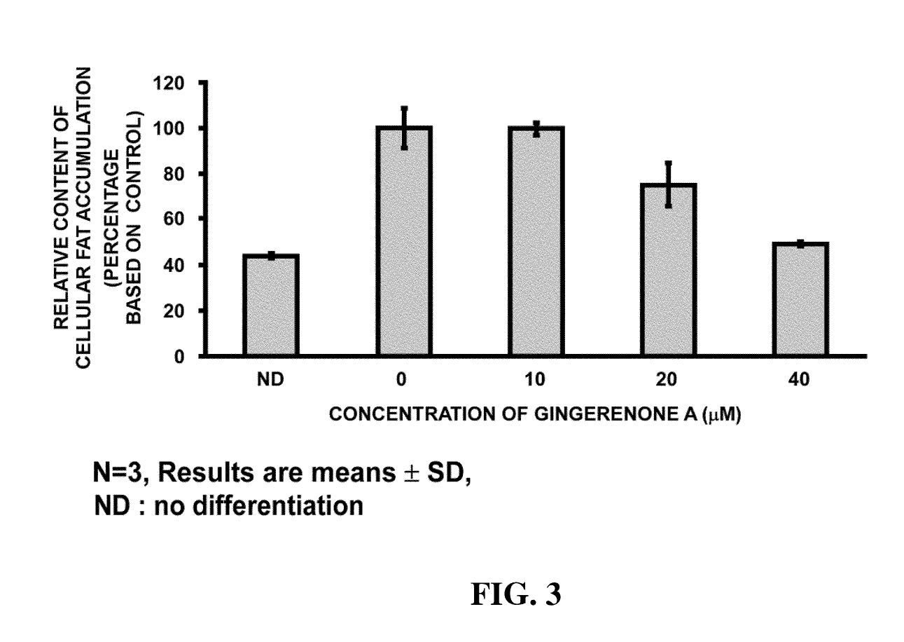

FIG. 3 shows the result of 3T3-L1 cells treated with gingerenone A. Cellular fat content decreased in the presence of gingerenone A treatment, which was dependent on the concentration of gingerenone A.

### Experimental Example 4

### Measurement of Suppressive Effects on C/EBPα, PPARγ and FAS Protein Expression by Gingerenone A

In Experimental Example 4, it was confirmed by Western blotting that gingerenone A affects important transcriptional factors, C/EBPα and PPARγ, and FAS protein expressed by a transcriptional factor thereof, expressed upon fat cell differentiation (B. L. Upham et al., Carcinogenesis. 18:37-42, 1997).

Post-confluent 3T3-L1 cells were cultured in DMEM supplemented with MDI, 10% FBS and 10 ml/L of antibiotic-antimycotic agent. In this media, we additionally treated with or without gingerenone A (10, 20 and 40 μM) for 2 days. For the next 2 to 4 days, the cells were further cultured. Here, the culture media was prepared by adding gingerenone A at concentrations of 10, 20 and 40 μM to DMEM media comprising insulin, 10% FBS and 1% antibiotic-antimycotic agent. For the next 4 to 6 days, the cells were further cultured in media comprising 10% FBS, 1% antibiotic-antimycotic agent and gingerenone A. Here, gingerenone A was added at concentrations of 10, 20 and 40 μM to the DMEM media. To extract protein from the cultured cells, after washing two times with cold PBS (phosphate buffered saline), the cells were lysed with a protein extraction solution. The protein extraction solution was RIPA buffer (50 mM, Tris pH 8.0, 150 mM NaCl, 1% NP-40, 5 mM EDTA and 1 mM PMSF) comprising 0.5% protease inhibitor (SIGMA-ALDRICH) and 0.5% phosphatase inhibitor (250 mM sodium fluoride, 5 mM sodium orthovanadate, 50 mM sodium pyrophosphate and 50 mM glycerophosphate). The cell lysates were centrifuged at 4° C. for 20 minutes at a rate of 12000 rpm. Supernatant formed by centrifugation was transferred to new 1.5 mL Eppendorf tubes. The protein content of the supernatants was determined using a DC assay kit (Bio-Rad Corp., Richmond, Calif., USA). The DC assay kit uses BSA (bovine serum albumin) as a standard solution. After mixing 20 to 50 μg of the protein with a sample buffer comprising bromophenol blue and denaturing the protein at 100° C. for 5 minutes, the denatured proteins were separated on a 12% SDS-polyacrylamide gel. After transferring the separated protein on the gel to a PVDF (polyvinylidene fluoride) membrane, the membrane was treated with blocking buffer (5% of skim milk, 94.8% of washing buffer and 0.2% of sodium azide) at room temperature for 1 hour. The reacted membrane was washed with a washing buffer for 10 minutes. The washing step was repeated three times. The washed membrane was incubated with PPARγ (purchased from Santa Cruz), C/EBPα (purchased from Cell Signal), FAS (purchased from Cell Signal) and β-actin (purchased from Sigma) antibodies at room temperature for 2 hours or at 4° C. overnight. The incubated membrane was washed with the washing buffer for 10 minutes. The washing step was repeated three times. The washed membrane was incubated with a secondary antibody (anti-rabbit IgG-HRP or anti-mouse IgG, Santa Cruz) which binds covalently with HRP (horseradish peroxidase). The incubated membrane was then washed with the washing buffer for 10 minutes. The washing step was repeated three times. After incubating the washed membrane with chemiluminescence solution (EZ-Western Detection Kit, Daeillab Service Co, Ltd) for 5 minutes, the incubated membrane was exposed to X-ray film. Finally, the X-ray film was developed.

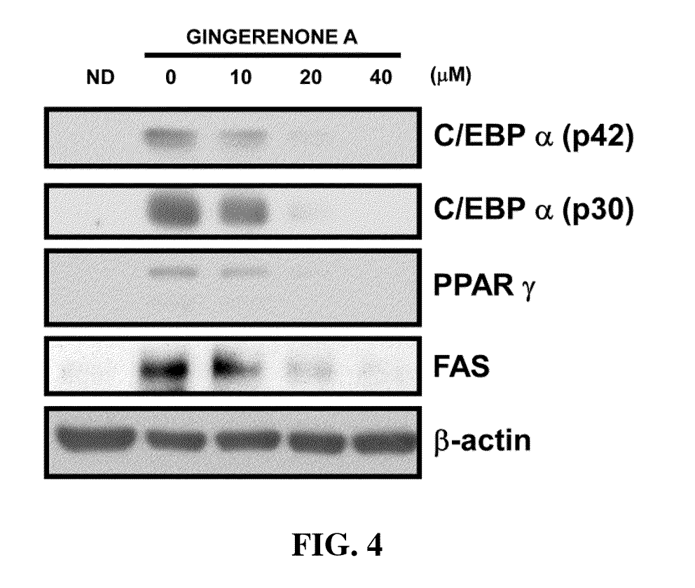

As a result of the Western blotting shown in FIG. 4, it was confirmed that C/EBPα, PPARγ and FAS protein expression levels were reduced by gingerenone A treatment.

### Experimental Example 5

### Comparison of Effects of Gingerenone A and Gingerol on Inhibition of C/EBPα, PPARγ and FAS Protein Expression

In Experimental Example 5, to compare the effects of gingerenone A and gingerol on the inhibition of C/EBPα, PPARγ and FAS expression, an experiment was carried out using the samples generated in Experimental Example 4. Here, an additional experiment was carried out with 40 μM gingerol.

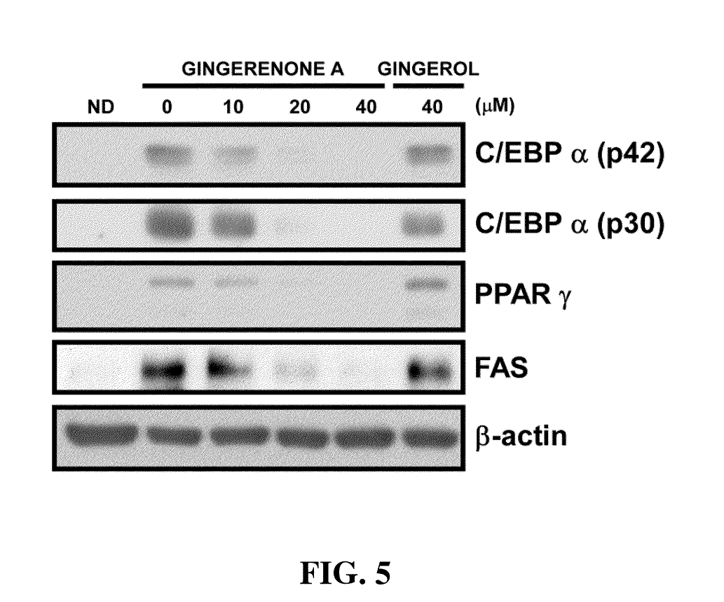

As shown in FIG. 5, C/EBPα and FAS expression levels were slightly reduced in samples treated with 40 μM gingerol whereas C/EBPα, PPARγ, and FAS expression levels were considerably reduced in samples treated with 40 μM gingerenone A. Thus, gingerenone A is considered as a material superior to gingerol for the prevention and treatment of obesity.

### Example 1

### Preparation of Food Composition for Preventing Obesity

A food composition for preventing obesity was prepared as follows.

(1) Preparation of Powder Composed of Mixed Grains

Brown rice, barley, glutinous rice and adlay were pre-gelatinized and dried according to a method described previously. Thereafter, the brown rice, barley, glutinous rice and adlay were roasted before being ground into powder with a particle size of 60 mesh using a grinder. Black bean, black sesame and perilla, respectively, were steamed and dried according to a method described previously. Thereafter, the black bean, black sesame and perilla were roasted before being ground into powder with a particle size of 60 mesh. Thereafter, the powder made of mixed grains was prepared by mixing 30% by weight of brown rice, 15% by weight of adlay, 20% by weight of barley, 9% by weight of glutinous rice, 7% by weight of perilla, 8% by weight of black bean, 7% by weight of black sesame, 3% by weight of gingerenone A, 0.5% by weight of Lingshi mushroom and 0.5% by weight of Rehmannia root.

(2) Preparation of Chewing Gum

Chewing gum was prepared by mixing 20% by weight of gum base, 76.9% by weight of sugar, 1% by weight of spice, 2% by weight of water and 0.1% by weight of gingerenone A, according to a conventional method.

(3) Preparation of Candy

Candy was prepared by mixing 60% by weight of sugar, 39.8% by weight of starch syrup, 0.1% by weight of spice and 0.1% by weight of gingerenone A, according to a conventional method.

(4) Preparation of Biscuits

Biscuits were prepared by mixing 25.59% by weight of first class weak flour, 22.22% by weight of first class medium flour, 4.80% by weight of refined sugar, 0.73% by weight of table salt, 0.78% by weight of glucose, 11.78% by weight of palm shortening, 1.54% by weight of ammonium, 0.17% by weight of baking soda, 0.16% by weight of sodium bisulfite, 1.45% by weight of rice flour, 0.0001% by weight of vitamin B, 0.04% by weight of milk flavor, 20.6998% by weight of water, 1.16% by weight of whole milk powder, 0.29% by weight of dried milk substitute, 0.03% by weight of calcium phosphate monobasic, 0.29% by weight of salt spray, 7.27% by weight of oil spray, and 1% by weight of gingerenone A, according to a conventional method.

(5) Preparation of Health Beverage

A health beverage was prepared by mixing 0.26% by weight of honey, 0.0002% by weight of thioctic acid amide, 0.0004% by weight of nicotinic acid amide, 0.0001% by weight of sodium riboflavin hydrochloride, 0.0001% by weight of pyridoxine hydrochloride, 0.001% by weight of inositol, 0.002% by weight of orotic acid, 98.7362% by weight of water and 1% by weight of gingerenone A, according to a conventional method.

(6) Preparation of Sausages

Sausages were prepared by mixing 65.18% by weight of pork, 25% by weight of chicken, 3.5% by weight of starch, 1.7% by weight of soybean protein, 1.62% by weight of table salt, 0.5% by weight of glucose, 1.5% by weight of glycerine, and 1% by weight of gingerenone A, according to a conventional method.

(7) Preparation of Health Supplements

Tablet-type health supplements were prepared by mixing 55% by weight of spirulina, 10% by weight of enzymatically decomposed guar gum, 0.01% by weight of vitamin B₁ hydrochloride, 0.01% by weight of vitamin B6 hydrochloride, 0.23% by weight of DL-methionine, 0.7% by weight of magnesium stearic acid, 22.2% by weight of lactose, 1.85% by weight of corn starch and 10% by weight of gingerenone A, according to a conventional method.

(8) Preparation of Alcoholic Beverage

To prepare an alcoholic beverage comprising gingerenone A, after mixing soju, beer, hard liquor or fruit wine with 0.5% by weight of gingerenone A, the mixed solution was centrifuged and mixed with a high speed mixer for 15 minutes at a rate of 7,000 rpm under vacuum.

### Example 2

### Preparation of Pharmaceutical Composition for Treating Obesity

A pharmaceutical composition for treating obesity was prepared as follows.

(1) Preparation of Powder

After mixing 2 g of gingerenone A and 1 g of lactose, the mixture was packed in an airtight package.

(2) Preparation of Tablet

To prepare a tablet, 100 mg of gingerenone A, 100 mg of corn starch, 100 mg of lactose and 2 mg of magnesium stearic acid were mixed and then compressed, according to a conventional method.

(3) Preparation of Capsule

To prepare a capsule, 100 mg of gingerenone A, 100 mg of corn starch, 100 mg of lactose and 2 mg of magnesium stearic acid were mixed before packing in a gelatin capsule.

(4) Preparation of Injectable Agent

To prepare an injectable agent, 100 mg of gingerenone A was dissolved in a specified quantity of distilled water for injection. The pH of the solution was adjusted to approximately 7.5. The solution was packed in a 2 ml ampoule. Finally, the ampoule was sterilized.

### Example 3

### Preparation of Medicine for Treating Animal Obesity

A medicine for treating animal obesity was prepared as follows.

(1) Preparation of Tablet

To prepare a tablet, 100 mg gingerenone A was mixed with 100 mg of protein mixture and 100 mg of wheat shorts and the mixture was compressed, according to a conventional method.

(2) Preparation of Injectable Agent

To prepare an injectable agent, 100 mg of gingerenone A was dissolved in a predetermined amount of distilled water. The pH of the solution was adjusted to approximately 7.5. Finally, the solution was packed in a 2 ml ampoule.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A food composition for preventing obesity comprising gingerenone A represented by the following Formula 1 as an active ingredient:

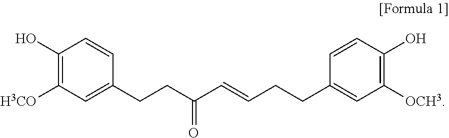 [SMILES: COc1cc(CC/C=C/C(=O)CCc2ccc(O)c(OC)c2)ccc1O]

2. The food composition according to claim 1, wherein the food composition comprises 0.000001 to 50% by weight of gingerenone A, based on 100% by weight in total of the food composition.

3. The food composition according to claim 1, wherein the food composition comprises at least one selected from meat, grains, caffeine beverages, beverages, chocolate, bread, snacks, biscuits, pizza, jelly, noodles, gums, ice cream, alcoholic beverages, vitamin complex and other health supplement foods.

4. A pharmaceutical composition for treating obesity comprising gingerenone A represented by the following Formula 1 as an active ingredient:

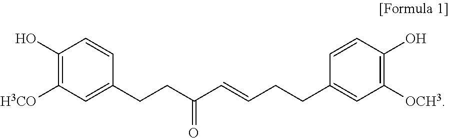 [SMILES: COc1cc(CC/C=C/C(=O)CCc2ccc(O)c(OC)c2)ccc1O]

5. The pharmaceutical composition according to claim 4, wherein the pharmaceutical composition comprises 0.000001 to 50% by weight of the gingerenone A, based on 100% by weight in total of the pharmaceutical composition.

6. The pharmaceutical composition according to claim 4, wherein the pharmaceutical composition comprises at least one selected from plasters, granules, lotions, liniments, lemonades, aromatic waters, powders, syrups, ophthalmic ointments, liquids, solutions, aerosols, extracts, elixirs, ointments, fluid extracts, emulsions, suspensions, decoctions, infusions, ophthalmic solutions, tablets, suppositories, injections, spirits, cataplasms, capsules, creams, troches, tinctures, pastes, pills, soft gelatin capsules and hard gelatin capsules.

7. A pharmaceutical composition for treating animal obesity comprising gingerenone A represented by the following Formula 1 as an active ingredient:

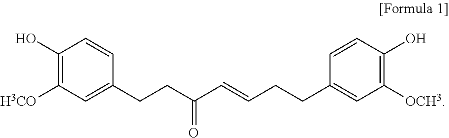 [SMILES: COc1cc(CC/C=C/C(=O)CCc2ccc(O)c(OC)c2)ccc1O]

8. The pharmaceutical composition according to claim 7, wherein the pharmaceutical composition comprises 0.000001 to 50% by weight of the gingerenone A, based on 100% by weight in total of the pharmaceutical composition.
